(19) United States
(12) Patent Application Publication
(10) Pub. No.: US 2005/0222052 A1
(43) Pub. Date: Oct. 6, 2005

(54) Process for preparing high purity azithromycin

(76) Inventors: Stefano Turchetta, Roma (IT); Pietro Massardo, Roma (IT); Paolo Casellato, Roma (IT)

Correspondence Address: WILLIAM COLLARD;COLLARD & ROE, P.C., 1077 NORTHERN BOULEVARD, ROSLYN, NY, 11576, US

(21) Appl. No.: 10/516,719

(22) PCT Filed: Jun. 4, 2003
(86) PCT No.: PCT/IB03/02442

§ 371 Date: Dec. 2, 2004

(30) Foreign Application Priority Data
Jun. 4, 2002 (IT) MI2002A001209

(51) Int. Cl. A61K 31/7052; C07H 17/08

(52) U.S. Cl. 514/28; 536/7.4

(57) ABSTRACT

A method for preparing high purity azithromycin is described characterised in that the intermediate 9a-deoxo-9a-aza-9a-homoerythromycin A is crystallised and obtained at very high purity, the subsequent methylation reaction effected on said intermediate proceeding with very high specificity and conversion enabling azithromycin of particularly high purity to be obtained.

### FIELD OF THE INVENTION

The present invention regards a process for preparing high purity azithromycin characterised in that the intermediate 9a-deoxo-9a-aza-9a-homoerythromycin A is crystallised and obtained at very high purity, the subsequent methylation reaction effected on said intermediate proceeding with very high specificity and conversion, enabling azithromycin of particularly high purity to be obtained.

### STATE OF THE ART

Azithromycin is an antibiotic which belongs to the macrolide class, with high activity against gram-positive and gram-negative bacteria.

It is synthesized from erythromycin A, which is a fermentation product, by following one of the two synthesis paths described in scheme 1 below.

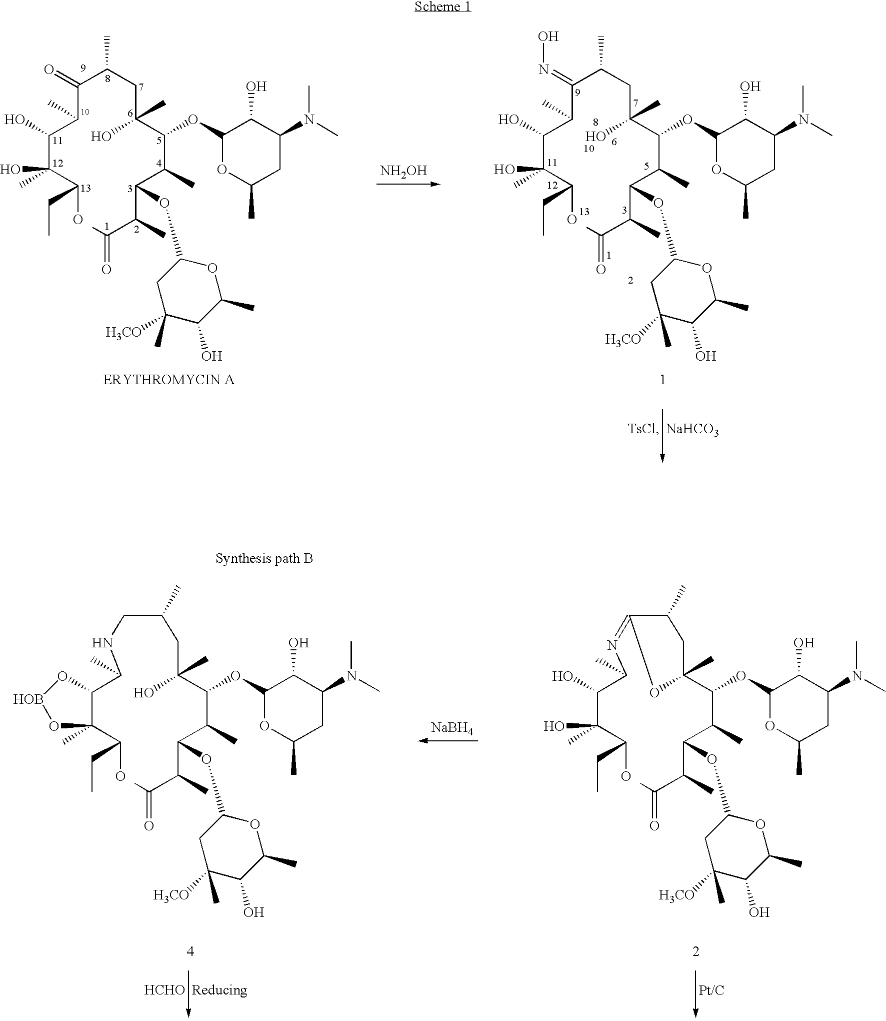

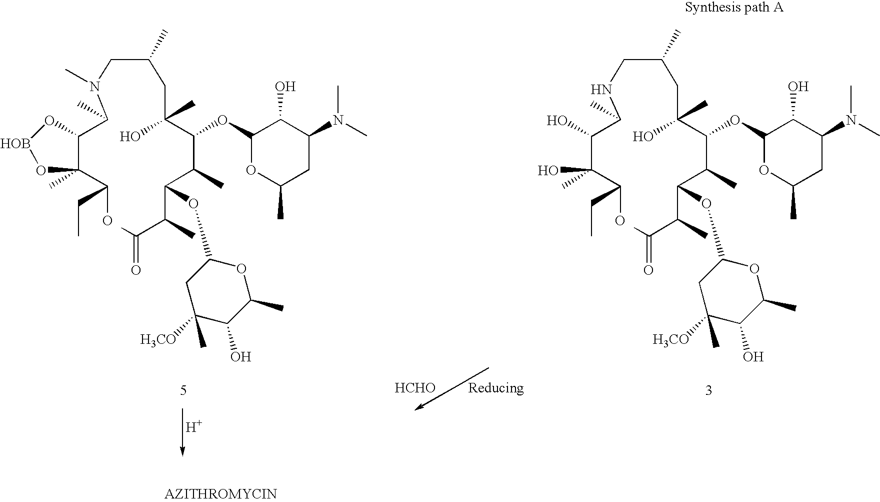

The synthesis path A is described for the first time in U.S. Pat. No. 4,328,334 and U.S. Pat. No. 4,517,359. EP827965 describes the hydrogenation of the iminoether (2) to 9a-deoxo-9a-aza-9a-homoerythromycin A (3) catalysed by Pt on carbon at 3-10 atm in a solvent consisting of a water-acetic acid-methanol mixture. EP879823 describes an abbreviated modification of the path A by effecting the reduction and methylation passage (from iminoether 2 to azithromycin) in a single stage. WO0210144 uses the synthesis scheme A to obtain a final compound in anhydrous crystalline form.

All the aforedescribed processes involving the synthesis path A comprise as intermediate the compound (3), which is not isolated, but is used directly as the reaction crude for the subsequent synthesis passages.

Another series of patents or patent applications relates to the application of the synthesis path (B) described in the aforesaid Scheme 1.

EP827965 describes and characterises the hydrogen orthoborate intermediates (4) and (5) of scheme 1. WO01100640 describes a method for effectively eliminating the hydrogen orthoborate group from the intermediate (5) by using polyhydroxylated solvents.

WO0215842 also uses the synthesis path B, isolating at the end of synthesis a crystalline form of anhydrous azithromycin.

Whatever the physical form in which the final product (crystalline monohydrate, dihydrate, crystalline anhydrous or amorphous azithromycin) is isolated, none of the processes described by the aforestated documents refers to synthesis techniques useful for improving the purity of the final product. Final product crystallisation is the only stated method which can serve for the purpose of purifying the product itself.

On the other hand, it is known that the purity of an active principle is a fundamentally important requirement for product quality as the impurities present can influence even to a very unfavourable extent the therapeutic effectiveness and the appearance of side effects, which can prejudice the use of the active principle in therapy.

The provision of a synthetic method enabling high purity azithromycin to be obtained would therefore be of considerable use.

### SUMMARY OF THE INVENTION

By applying the synthesis path A described in Scheme 1 for azithromycin synthesis, it has been surprisingly found that the product (3) can be obtained in crystalline form with high purity. The subsequent Eschweiler-Clarke methylation reaction on this product proceeds with high specificity, leading to the formation of very high purity azithromycin.

The present invention therefore provides a process for preparing high purity azithromycin comprising the following stages:

a) hydrogenating the iminoether (2) with Pt/C to obtain 9a-deoxo-9a-aza-9a-homoerythromycin A (3),

b) methylating the 9a-deoxo-9a-aza-9a-homoerythromycin A originating from stage (a) with formaldehyde and formic acid,

characterised in that stage (a) is conducted in water to which acids have been previously added until a pH≧4 is obtained and once the reaction is completed the 9a-deoxo-9a-aza-9a-homoerythromycin A being isolated by crystallisation.

The present invention therefore further provides 9a-deoxo-9a-aza-9a-homoerythromycin A, in crystalline form which on X-ray diffraction at wavelength Kα presents the image defined by the following table:

TABLE 1

| | | Relative |
|---|---|---|
| Angle 2 θ | d (Å) | intensity (I/Iₒ) |
| 7.285 | 12.125 | 100.0 |
| 11.290 | 7.831 | 57.5 |
| 12.595 | 7.022 | 64.9 |
| 14.590 | 6.066 | 58.0 |
| 18.405 | 4.817 | 61.0 |
| 19.320 | 4.590 | 40.2 |
| 21.005 | 4.226 | 32.3 |
| 22.355 | 3.974 | 35.0 |
| 22.800 | 3.897 | 38.3 |
| 29.630 | 3.762 | 31.7 |

### DESCRIPTION OF THE FIGURES

FIG. 1 shows the XRD spectrum in which the vertical axis represents the count number and the horizontal axis the values of the angle 2θ.

FIG. 2 shows the IR spectrum of 9a-deoxo-9a-aza-9a-homoerythromycin A in crystalline form.

FIG. 3 shows the relative 1H-NMR spectrum.

FIG. 4 shows the relative 13C-NMR spectrum.

### DETAILED DESCRIPTION OF THE INVENTION

Stage (a) of the process of the present invention presents a further advantage, namely that it is conducted using only acidified water as solvent, hence under much more favourable conditions than those described in the literature, which use as reaction solvent glacial acetic acid (EP879823 and U.S. Pat. No. 4,328,334) or mixtures of water, alcohols and acetic acid (EP827965). In this respect the iminoether (2) is unstable both in glacial acetic acid and in acetic acid-water-alcohol mixtures, giving rise to extended impurity formation to the detriment both of the yield and the purity of the product obtained by the hydrogenation. Moreover, water-alcohol mixtures are found to lead to rapid degradation of the secondary amine (3), the product of the hydrogenation reaction.

Stage (a) is preferably effected after solubilizing the iminoether in water at 5° C. by adding an acid until reaching a pH not less that 4.0, preferably between 4 and 6. The acid to be added can be chosen from hydrochloric acid, sulphuric acid, phosphoric acid, methanesulphonic acid, acetic acid, formic acid. Phosphoric acid is preferably used.

The iminoether solution hence obtained is sufficiently stable to be able to be hydrogenated. The amount of catalyst used in stage (a) can vary between 50 and 10% on the weight of the iminoether fed. 5% Pt/C 50% wet, is preferably used. The hydrogenation is preferably effected at a pressure between 10 and 40 bar, more preferably between 15 and 25 bar and even more preferably at 20 bar for a time period between 12 and 24 hours at a temperature between 0 and 20° C., more preferably between 10 and 15° C.

Separation of the crystalline form of 9a-deoxo-9a-aza-9a-homoerythromycin A by crystallisation, a further aspect of the present invention, is preferably effected by a method comprising the following stages:

i) the catalyst is eliminated by filtration and the reaction mixture is treated with an organic solvent immiscible with water and then with bases possibly dissolved in an aqueous solution, the product is extracted, and the solvent evaporated,

ii) the product originating from the preceding stage is dissolved in a solvent miscible with water, after which water is added in a quantity between 1 and 100 volumes/volume of organic solvent at a temperature between −20 and +50° C., to obtain a suspension,

iii) the suspension is left under stirring for a time between 1 and 12 hours,

iv) the product is filtered, washed with water and dried in an oven at 40° C. under vacuum at 40 mm Hg for 12 hours.

The base used in stage (i) of the crystallisation method of the present invention is an inorganic base preferably chosen from NaOH, KOH, Na₂CO₃, K₂CO₃ and ammonia or an organic base such as triethylamine, whereas the organic solvent used in said stage of the method for crystallising 9a-deoxo-9a-aza-9a-homoerythromycin A in crystalline form is usually chosen from hydrocarbons, ethers, esters, chlorinated solvents; preferably it is chosen from cyclohexane, toluene, ethyl acetate, isopropyl acetate, ethyl ether, isopropyl ether, methyl tert-butylether, dichloromethane.

In stage (ii) of the crystallisation method of the present invention, acetone is preferably used as the organic solvent miscible with water; in this case the quantity of water to be added to said solvent is preferably twice the volume of said solvent.

The temperature at which stage (iii) is conducted is preferably between 20 and 25° C.

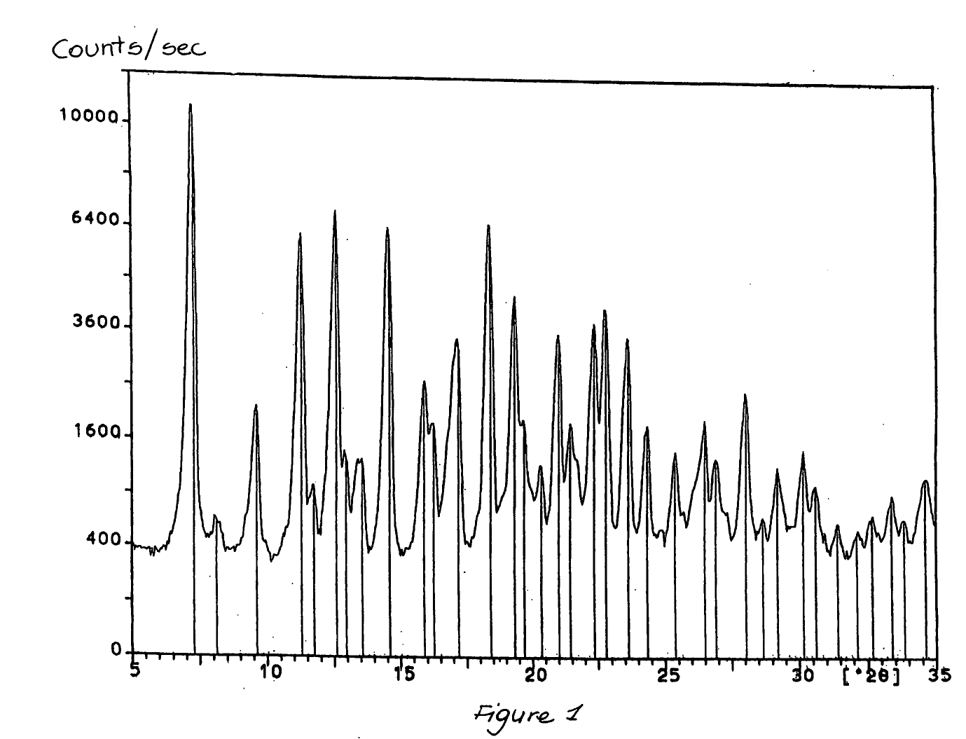

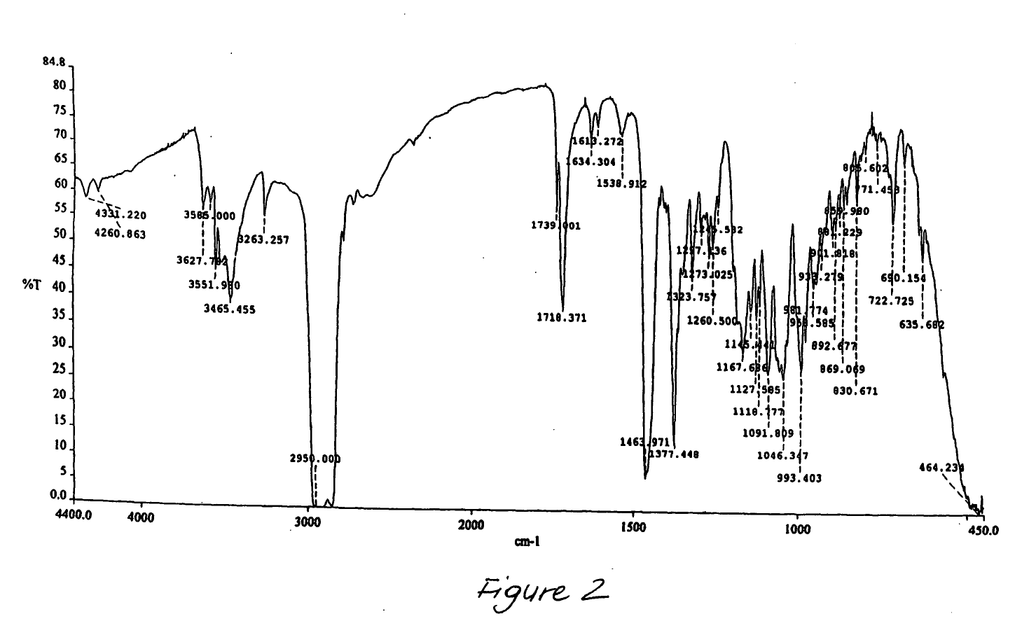

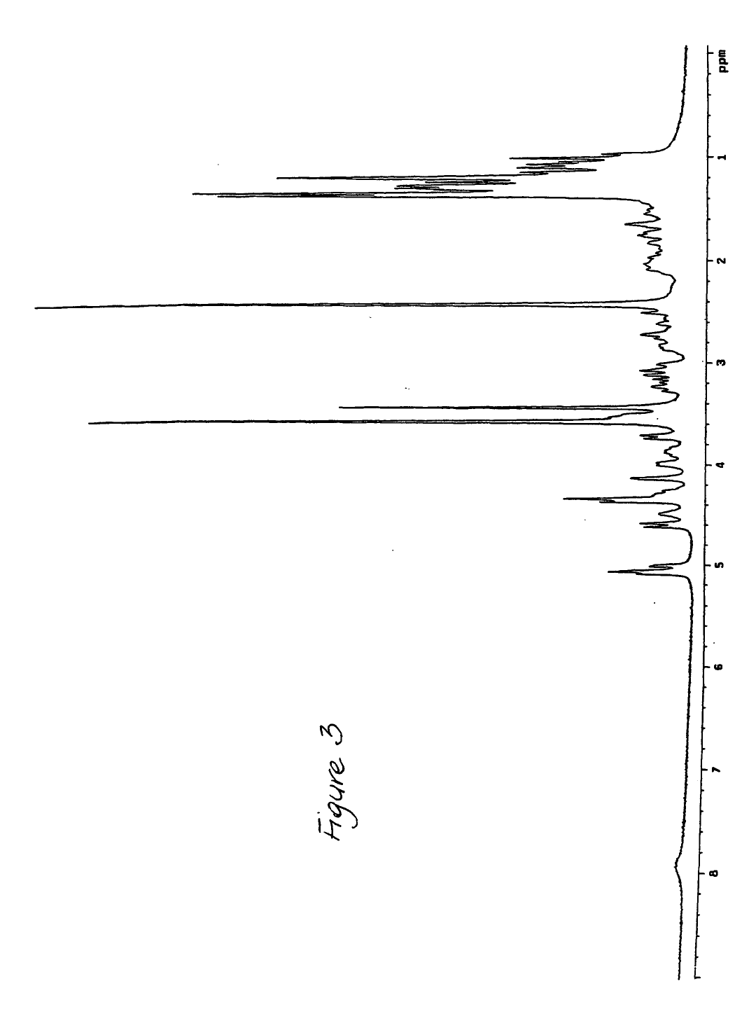

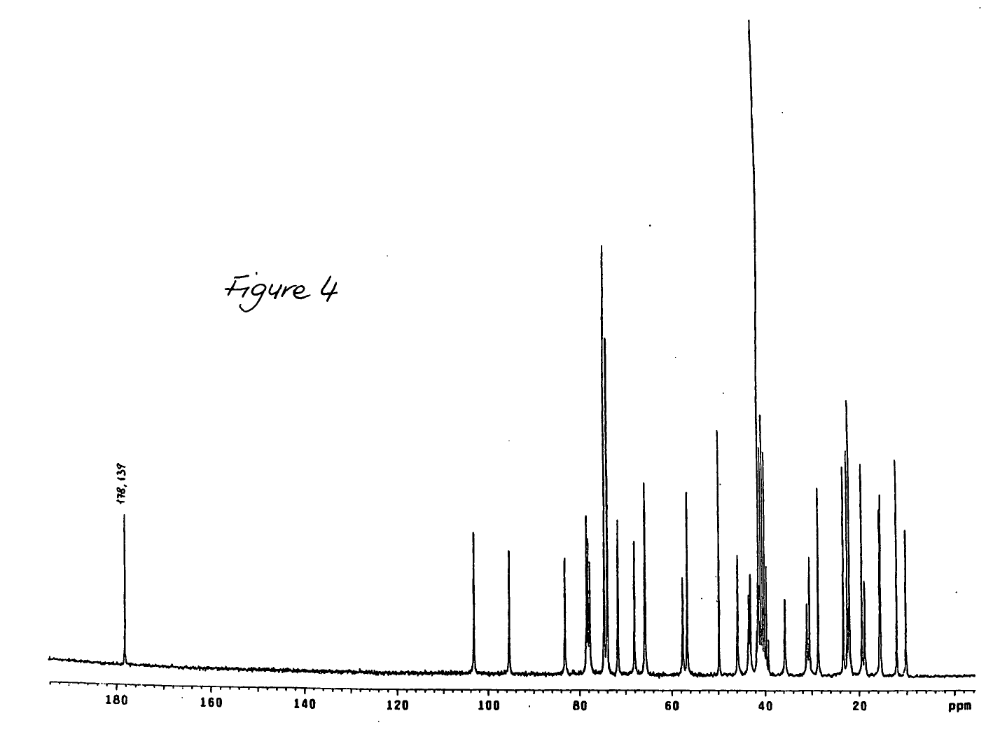

The 9a-deoxo-9a-aza-9a-homoerythromycin A in crystalline form presents the XRD spectrum indicated in FIG. 1, in which the values of the angle 2θ and the distance d (Å) of the most relevant peaks are reported in said table 1, the IR spectrum is reported in FIG. 2, the 1H-NMR spectrum in FIG. 3, the 13C-NMR spectrum in FIG. 4.

The crystalline product yield is 78-80%. The crystalline product obtained is then converted into azithromycin by methylation in accordance with the Eschweiler-Clarke method, as described in the literature. Specifically, the crystalline product is dissolved in organic solvent such as isopropyl acetate, acetone, dichloromethane or acetonitrile, isopropyl acetate preferably being used, to the solution there then being added formaldehyde in the form of paraformaldehyde, trioxane or a 30% aqueous formaldehyde solution; optionally triethylamine is added to the mixture. Formic acid is then added. The mixture thus obtained is heated to reflux temperature and maintained in that state for a time period between 2 and 16 hours, preferably for 4 hours, after which the mixture is cooled, water added and treated with bases. The phases are separated and the aqueous phase is re-extracted with organic solvent. The organic extracts containing crude azithromycin are pooled and evaporated to dryness and then dissolved in ethanol. Finally the ethanol solution is brought to 40-50° C. and water slowly added as described in U.S. Pat. No. 4,474,768. In this manner crystalline azithromycin monohydrate precipitates, is filtered, washed with water and dried at 40° C. for 12 hours under a residual pressure of 40-50 mm Hg.

On TLC and HPLC analysis, the crystalline product obtained shows an impurity framework decidedly lower than that of commercial samples of the same product.

The following examples are given as non-limiting illustration of the process for preparing high purity azithromycin according to the present invention.

### EXAMPLE 1

Preparation of 9a-deoxo-9a-aza-9a-homoerythromycin A in Crystalline Form

156 g of iminoether (2), calculated as anhydrous products, and 1240 ml of deionised water are fed into a 2 litre reactor fitted with mechanical stirrer. The suspension is brought to 5° C. and 35.3 ml of 85% phosphoric acid are added to it, observing the dissolution of the product, the solution pH obtained in this manner being between 4 and 4.5.

78 g of 5% Pt/C, wetted to 50% wet, are fed into a 3 litre autoclave vessel followed by the previously prepared iminoether solution. The suspension temperature is brought to 15° C., then after effecting 3 vacuum-nitrogen scavenging cycles, the reactor is fed with hydrogen at a pressure of 20 bar. It is left under these conditions for 24 hours, at the end of which the hydrogenator is purged by effecting 3 vacuum-nitrogen scavenging cycles. The catalyst is then filtered and 300 ml of isopropyl acetate are added to the resultant solution, which is then treated with 110 ml of 30% sodium hydroxide. The organic phase is removed and the aqueous phase is again extracted with 300 ml of isopropyl acetate.

The pooled organic phases are evaporated to residue and redissolved in 320 ml of acetone, to obtain a solution. 640 ml of deionised water are then added slowly to the solution to progressively render the mixture turbid until a heavy crystalline product is precipitated.

The crystalline product is left to mature at ambient temperature for 4 hours, after which the solid is filtered off and washed with 200 ml of deionised water. The product is discharged and dried at 40° C. for 12 hours under a residual pressure of 400 mm Hg and consists of crystalline 9a-deoxo-9a-aza-9a-homoerythromycin A, which on X-ray diffraction at wavelength Kα presents the image defined by said table 1 and FIG. 1, the IR, 1H-NMR, 13C-NMR spectra being reported respectively in FIGS. 2-4.

### EXAMPLE 2

Preparation of Crystalline Azithromycin Monohydrate

100 g of crystalline 9a-deoxo-9a-aza-9a-homoerythromycin A obtained as described in the preceding example, 9.2 g of paraformaldehyde, 44.9 ml of triethylamine and 603 ml of isopropyl acetate are fed into a 2 litre reactor equipped with a mechanical stirrer, condenser and thermometer. The mixture is then brought to 50° C. and 12.2 ml of formic acid are added. The heterogeneous mixture is heated to 70° C. and maintained under these conditions for 4 hours, at the end of which it is cooled to ambient temperature and 320 ml of de-ionised water are added to the mixture, which is treated with 12.8 ml of 30 sodium hydroxide.

The phases are separated and the aqueous phase is again extracted with 192 ml of isopropyl acetate. The pooled organic extracts are then evaporated to dryness and redissolved in 225 ml of absolute ethanol. 675 ml of deionised water are slowly added to the solution obtained, which is brought to 50° C., observing the progressive turbidity of the mixture, which over time gives rise to a suspension of crystalline material. The mixture is maintained at 20-25° C. for 4 hours, then filtered and washed with 130 ml of deionised water. The crystalline solid is discharged and dried at 40° C. for 12 hours under a residual pressure of 40 mm Hg. The dry solid consisting of crystalline azithromycin weighs 96.1 g (yield 95%). The spectroscopic data (IR, NMR, XRD) and the spectrum confirm that this is crystalline azithromycin monohydrate. TLC and HPLC analyses confirm that the azithromycin monohydrate obtained in this example presents a greater purity than the corresponding product in monohydrate form obtained as described in U.S. Pat. No. 4,474,768.

1. Process for preparing high purity azithromycin comprising the following stages:
a) hydrogenating the iminoether (2) with Pt/C to obtain 9a-deoxo-9a-aza-9a-homoerythromycin A,
b) methylating the 9a-deoxo-9a-aza-9a-homoerythromycin A (3) originating from stage (a) with formaldehyde and formic acid,
wherein the stage (a) is conducted in water to which acids have been added until a pH≧4 is attained, the 9a-deoxo-9a-aza-9a-homoerythromycin A being isolated by crystallisation at the end of the reaction.

2. Process as claimed in claim 1, wherein the stage (a) is carried out after dissolving the iminoether at 5° C. in water by adding an acid until a pH not less than about 4 is obtained.

3. Process as claimed in claim 2, wherein the pH is between about 4 and about 6.

4. Process as claimed in claim 1, wherein said acid is phosphoric acid.

5. Process as claimed in claim 1, wherein the stage (a) is conducted at a pressure between about 10 and about 40 bar.

6. Process as claimed in claim 5, wherein said pressure is between about 15 and about 25 bar.

7. Process as claimed in claim 1, wherein the stage (a) is conducted at a temperature between about 0 and about 20° C.

8. Process as claimed in claim 7, wherein the stage (a) is conducted at a temperature between about 10 and about 15° C.

9. Process as claimed in claim 1, wherein the separation of the crystalline form of 9a-deoxo-9a-aza-9a-homoerythromycin A by crystallisation is effected by a method which comprises the following stages:
i) the catalyst is eliminated by filtration and the reaction mixture is treated with an organic solvent immiscible with water and then with bases possibly dissolved in an aqueous solution, the product is extracted, and the solvent evaporated,
ii) the product originating from the preceding stage is dissolved in a solvent miscible with water, after which water is added in a quantity between about 1 and about 100 volumes/volume of organic solvent at a temperature between about −20 and +50° C., to obtain a suspension,
iii) the suspension is left under stirring for a time between 1 and 12 hours,
iv) the product is filtered off, washed with water and dried in an oven at about 40° C. under vacuum at 40 mm Hg for 12 hours.

10. Process as claimed in claim 9, in stage (i) of the crystallisation method, the base is chosen from NaOH, KOH, Na₂CO₃, K₂CO₃, ammonia and triethylamine.

11. Process as claimed in claim 9, wherein said organic solvent immiscible in water is chosen from the group consisting of cyclohexane, toluene, ethyl acetate, isopropyl acetate, ethyl ether, isopropyl ether, methyl tert-butylether, dichloromethane.

12. Process as claimed in claim 9, wherein in stage (ii) acetone is used as the crystallisation solvent, the water being added to the extent of about 2 volumes/volume of acetone.

13. Process as claimed in claim 9, wherein the temperature is between about 20 and about 25° C.

14. 9a-deoxo-9a-aza-9a-homoerythromycin A, in crystalline form, which under X-ray diffraction at the wavelength Kα presents the image defined by the following table:
TABLE 1

| | | Relative |
|---|---|---|
| Angle 2 θ | d (Å) | intensity (I/Iₒ) |
| 7.285 | 12.125 | 100.0 |
| 11.290 | 7.831 | 57.5 |
| 12.595 | 7.022 | 64.9 |
| 14.590 | 6.066 | 58.0 |
| 18.405 | 4.817 | 61.0 |
| 19.320 | 4.590 | 40.2 |
| 21.005 | 4.226 | 32.3 |
| 22.355 | 3.974 | 35.0 |
| 22.800 | 3.897 | 38.3 |
| 29.630 | 3.762 | 31.7 |
